(19) United States
(12) Patent Application Publication
(10) Pub. No.: US 2025/0327006 A1
(43) Pub. Date: Oct. 23, 2025

(54) APPARATUS FOR PRODUCING BIOMETHANE AND METHOD OF PRODUCING BIOMETHANE FROM WASTE USING THE SAME

(71) Applicant: BIO X CO., LTD., Seoul (KR)

(72) Inventor: Dae Yeol CHEONG, Seoul (KR)

(73) Assignee: BIO X CO., LTD., Seoul (KR)

(21) Appl. No.: 18/961,140

(22) Filed: Nov. 26, 2024

Related U.S. Application Data
(63) Continuation of application No. PCT/KR2024/005231, filed on Apr. 18, 2024.

(51) Int. Cl. C12M 1/107; C12M 1/00; C12N 1/20; C12P 5/02; C12R 1/145; C12R 1/46

(52) U.S. Cl. CPC C12M 21/04; C12M 23/58; C12M 29/00; C12N 1/20; C12P 5/023; C12R 2001/145; C12R 2001/46

(57) ABSTRACT

An apparatus for producing biomethane and a method for producing biomethane from waste using the apparatus are provided. The apparatus includes a first reaction tank configured to produce, from a liquid phase including wastewater, a fermentation product including an organic acid. The apparatus further includes a second reaction tank configured to produce, from a gas phase, a fermentation product including an organic acid. The apparatus further includes a third reaction tank configured to produce methane from the fermentation product derived from the first reaction tank and the second reaction tank.

### TECHNICAL FIELD

The present invention relates to an apparatus for producing biomethane and a method for producing biomethane from waste using the same.

### BACKGROUND

Fossil fuels are one of the most commonly used resources for energy production. However, fossil fuels have limited reserves, and when burned, they emit fine particles, carbon monoxide, carbon dioxide, sulfur and nitrogen oxides, and the like, and are considered a major contributor to environmental pollution. Therefore, research on energy sources that can replace fossil fuels is actively being carried out, and biogas is gaining attention as a clean energy source.

Biogas is a gas obtained by digesting organic waste with a high content of biomass, such as livestock excreta, food waste, and wastewater treatment facility sludge, under anaerobic conditions where oxygen is scarce. The main components of biogas are methane, which accounts for about 60 to 70 percent, and carbon dioxide, which accounts for about 30 to 40 percent, with trace amounts of hydrogen sulfide and ammonia. Among these, methane has a high potential to be used as an alternative to coal and oil, and can be easily converted to hydrogen, but it has the problem of causing global warming when emitted into the air. Therefore, the relevant industry has been developing technologies to separate methane from biogas, and recently, research activities have gained attention on technologies to produce biomethane with high efficiency.

Technologies for separating biomethane, such as a water scrubbing method, which uses the solubility differences between gases to separate methane and carbon dioxide, a pressure swing adsorption (PSA) method, which performs separation using the difference in adsorption properties of gas-phase materials, and a membrane separation method, which uses the polarity differences of gas molecules, are well-known. However, these technologies have clear limitations such as high maintenance and poor economic viability. As a result, the development of these corresponding technologies has recently been in decline. In this regard, the development of biomethane with high purity has received relatively more attention in recent years.

In the methods of producing biomethane, there are largely physicochemical methods and biological methods, and in case of the former, there is the disadvantage of requiring a large amount of energy sources to maintain high temperature and high pressure conditions during production. Meanwhile, the biological method is a process that only uses light energy, water, organic matter, and microorganisms, making it an environmentally friendly method, and is therefore preferred over the physicochemical method. However, the biological method faces technical limitations, such as high initial facility investment costs and low biogas production efficiency.

### DOCUMENT OF RELATED ART

(Patent Document 0001) Korean Patent No. 10-1544097

Acknowledgement: This invention was supported by the Energy Conversion Demonstration Technology Development Project Using Waste Resources, funded by the Ministry of Economy and Finance and the Korea Environmental Industry & Technology Institute. [1485019393, ARQ202201657002, Development of Demonstration Technology for Energy Conversion Using Unused Complex Biomass, 2022 Apr. 1˜2022 Dec. 31, Lead Organization: Samchully].

### Technical Problem

In one aspect, an object of the present invention is to produce biomethane from waste.

In one aspect, an object of the present invention is to produce biomethane of high purity from waste.

In one aspect, an object of the present invention is to produce biomethane of high purity with high efficiency.

### Technical Solution

In order to achieve the aforementioned object, the present invention is directed to providing an apparatus for producing biomethane, including a first reaction tank that produces, from a liquid phase including wastewater, a fermentation product including an organic acid, a second reaction tank that produces, from a gas phase, a fermentation product including an organic acid, and a third reaction tank that produces methane from the fermentation product derived from the first reaction tank and the second reaction tank.

The apparatus for producing biomethane may further include a microbial culture device configured to culture a microorganism and to feed the cultured microorganisms to the first reaction tank.

The microorganism may produce acetic acid or ethanol from carbon dioxide or produces hydrogen under anaerobic conditions.

The microorganism producing acetic acid or ethanol from carbon dioxide may be formed from at least one microorganism selected from the group consisting of Clostridium, Enterobacter, Lactobacillus, Escherichia, Ruminococcus, Propionibacterium, Acetobacterium, Butyribacterium, Streptococcus, and the like.

The microorganism producing hydrogen under anaerobic conditions may be formed from at least one microorganism selected from the group consisting of Clostridium, Sporanaerobacter, Coprococcus, Enterococcus, Morella, Thermoanaerobacterium, and Enterobacter.

A fermentation product in gas phase of the fermentation products produced in the first reaction tank may be introduced into the second reaction tank.

The gas phase may include at least one of hydrogen or carbon dioxide.

The apparatus for producing biomethane may further include a mixing tank configured to mix a fermentation product produced in the first reaction tank with a fermentation product produced in the second reaction tank.

The third reaction tank may perform anaerobic fermentation using a microorganism on the fermentation products produced from the first reaction tank and the second reaction tank.

The microorganism used in the third reaction tank may be formed from at least one microorganism selected from the group consisting of Methanobacterium, Methanococcus, Methanosarcina, Methanothhrix, Methanolobus, Methanococcoides, or Methnoplanus.

An internal temperature of the third reaction tank may be formed from 20 to 60° C.

The apparatus for producing biomethane may further include a fourth reaction tank, and a hydrogen feed tank, in which a gas phase of the fermentation products produced in the third reaction tank may be introduced into the fourth reaction tank, and the hydrogen feed tank may feed hydrogen to the fourth reaction tank.

The fourth reaction tank may include methane-producing bacteria.

The methane-producing bacteria may be a microorganism capable of producing methane from hydrogen and formed from at least one microorganism selected from the group consisting of Methanobacterials, Methanococcales, Methanomicrobials, or Methanosarcinaceae.

An internal temperature of the fourth reaction tank may be formed from 55 to 80° C.

The hydrogen feed tank may feed hydrogen so that a molar fraction (hydrogen:carbon dioxide) of hydrogen and carbon dioxide present in the fourth reaction tank is 3 to 5:1.

The apparatus for producing biomethane may further include a microbubble generator coupled to the fourth reaction tank and configured to feed a gas in the form of microbubbles to an interior of the fourth reaction tank.

The organic acid may include acetic acid.

Further, disclosed herein is a method of producing biomethane from waste, using the aforementioned apparatus for producing biomethane.

In addition, disclosed herein a system for producing biomethane. The system may include the aforementioned apparatus for producing biomethane, configured to produce biomethane; and a sludge storage tank configured to store sludge generated from the apparatus for producing biomethane, filter, circulate and feed an unused portion of the sludge, or feed organic matter reserves required for methane gas production.

### Advantageous Effects

An apparatus for producing biomethane, which is one aspect of the present invention, is a technology for producing biomethane from discarded wastewater, which is eco-friendly and economical. In addition, the apparatus for producing biomethane according to the present invention can use carbon materials such as carbon dioxide present in the atmosphere, thereby contributing to carbon neutrality. In addition, the apparatus for producing biomethane according to the present invention has a high biomethane production efficiency by performing a liquid-phase fermentation process separately.

### DESCRIPTION OF DRAWINGS

FIGS. 1 to 9 are embodiments of an apparatus for producing biomethane, which is one aspect of the present invention.

FIG. 10 illustrates an example of an anaerobic metabolic pathway for the decomposition of organic matter.

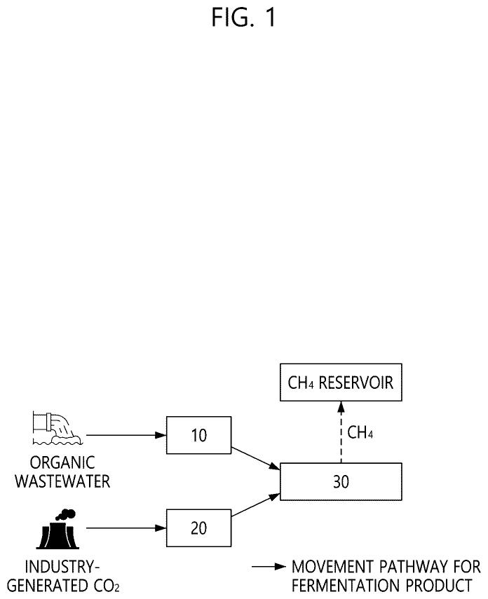

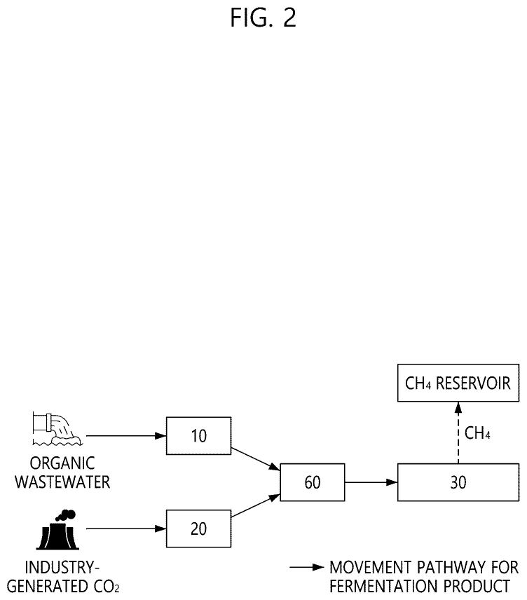

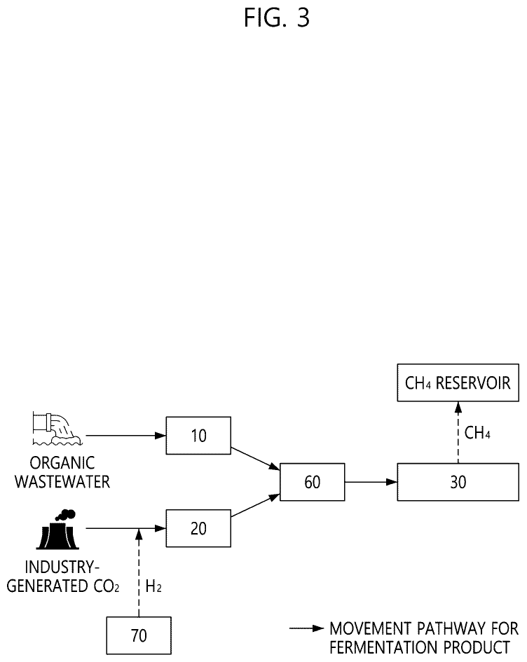

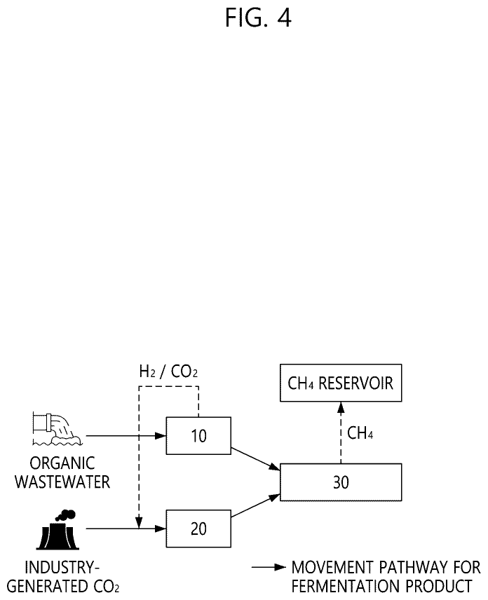

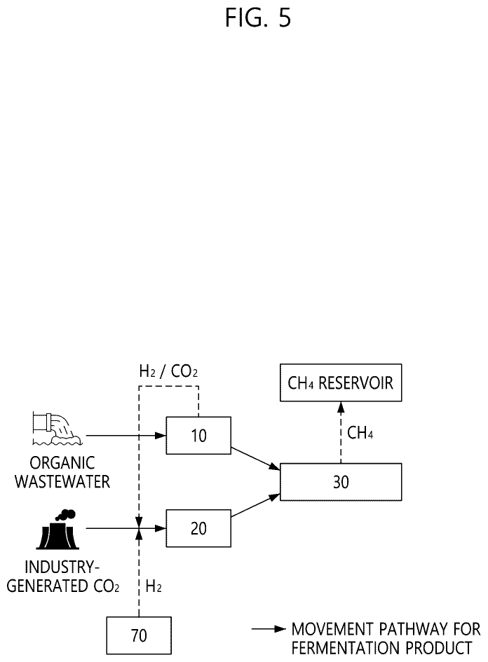

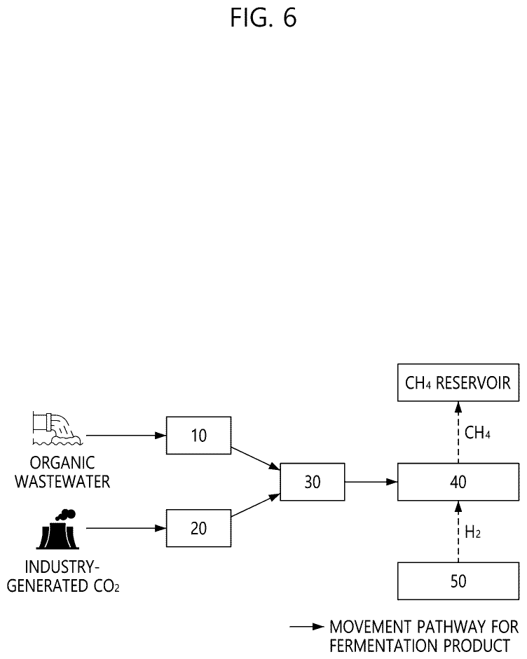

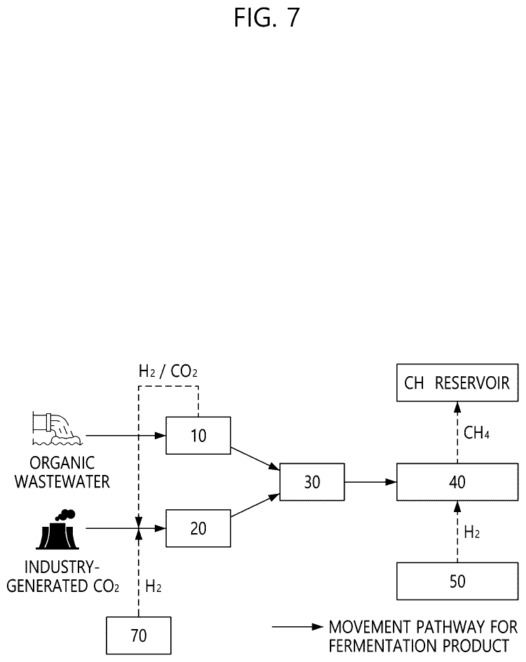

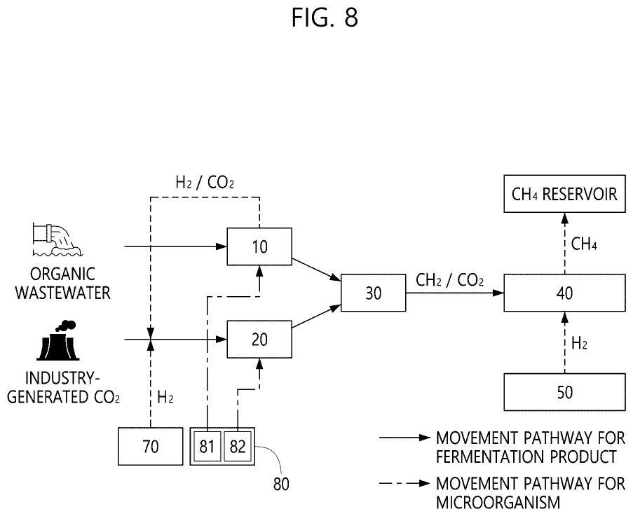

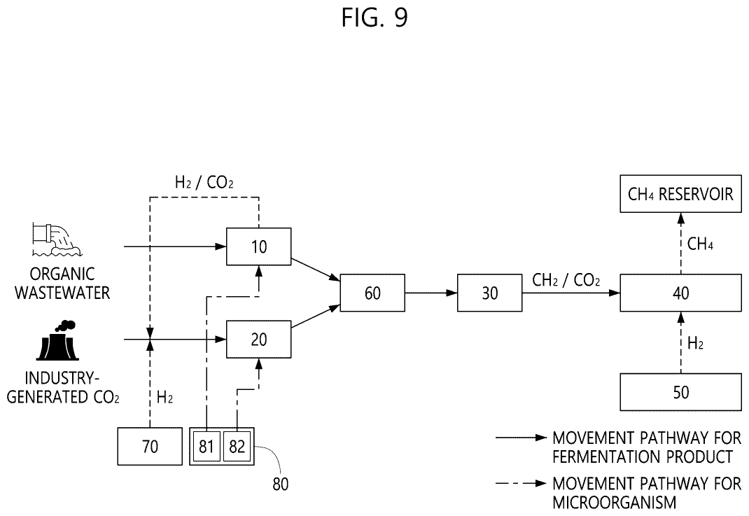

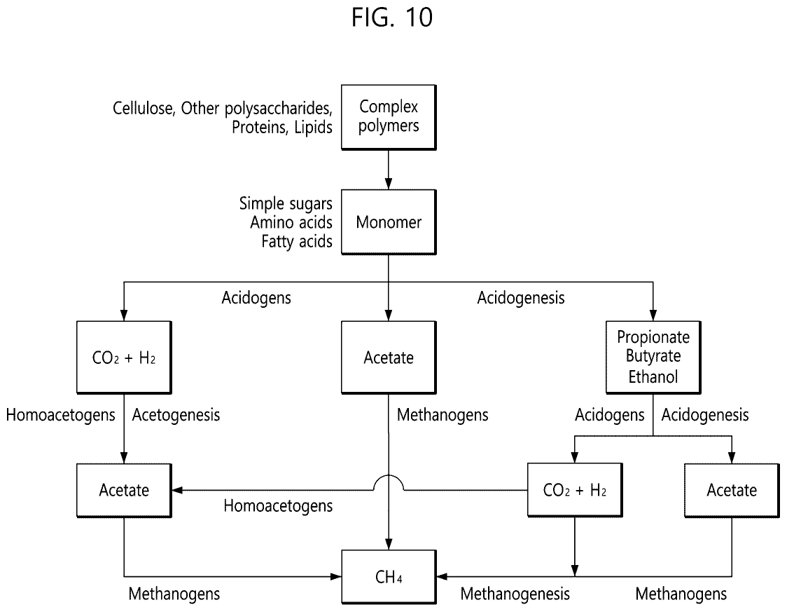

### DETAILED DESCRIPTION OF EMBODIMENTS

The present invention is based on the result of research conducted under the support of the Development of demonstration technology project for energy conversion using waste resource of the Korea Environmental Industry and Technology Institute under the Ministry of Economy and Finance (tax number: ARQ202201657001, project name: Development of demonstration technology for energy conversion using unused complex biomass, research period: 2022 Apr. 1˜2022 Dec. 31.

Hereinafter, exemplary embodiments of the present invention will be described in detail with reference to the accompanying drawings so that those with ordinary skill in the art to which the present invention pertains may easily carry out the exemplary embodiments. However, the present invention may be implemented in various different ways and is not limited to the embodiments described herein. Further, a part irrelevant to the description will be omitted in the drawings in order to clearly describe the present invention, and similar constituent elements will be designated by similar reference numerals throughout the specification.

Terms or words used in the specification and the claims of the present invention should not be interpreted as being limited to a general or dictionary meaning and should be interpreted as a meaning and a concept which conform to the technical spirit of the present invention based on a principle that an inventor can appropriately define a concept of a term in order to describe his/her own invention by the best method.

Throughout the specification of the present invention, unless explicitly described to the contrary, the word “comprise” or “include” and variations, such as “comprises”, “comprising”, “includes” or “including”, means the further inclusion of stated constituent elements, not the exclusion of any other constituent elements.

Throughout the specification of the present invention, “A and/or B” refers to A or B, or A and B.

In addition, temperature units in this specification are in degrees Celsius (° C.) unless otherwise noted.

Hereinafter, the present invention will be described in detail with reference to the accompanying drawings, but the present invention is not limited thereto.

In one aspect, the present invention relates to an apparatus for producing biomethane, including a first reaction tank 10 that produces, from a liquid phase including wastewater, a fermentation product including an organic acid, a second reaction tank 20 that produces, from a gas phase, a fermentation product including an organic acid, and a third reaction tank 30 that produces methane from the fermentation product derived from the first reaction tank and the second reaction tank.

As used herein the term “fermentation product” refers to all materials produced during fermentation and after completion of fermentation, including intermediate products of fermentation and end products of fermentation.

In one aspect, the wastewater may include organic wastewater. The organic wastewater may refer to wastewater with a high content of organic matter and may include, for example, livestock excreta, food wastewater, and the like, but is not limited thereto.

In one aspect, in the first reaction tank, fermentation (liquid fermentation) is performed using microorganisms, with wastewater, which is in a liquid phase, as a substrate. In this case, organic acids such as acetic acid are produced in the first reaction tank (acetogenesis), and gases or gas phases such as carbon dioxide and hydrogen are also produced.

In one aspect, the organic acid may include acetic acid, butyric acid, stearic acid, propionic acid, citric acid, and the like.

In one aspect, the microorganisms used in the first reaction tank may include at least one of Clostridium, Enterobacter, Lactobacillus, Escherichia, Ruminococcus, Propionibacterium, Acetobacterium, Butyribacterium, or Streptococcus.

In addition, the microorganisms used in the first reaction tank may be used, without limitation, as long as they are capable of producing acetic acid and/or ethanol from carbon dioxide.

In addition, the microorganisms used in the first reaction tank may include microorganisms capable of producing hydrogen. For example, the microorganisms used in the first reaction tank may include one at least one of Clostridium, Sporanaerobacter, Coprococcus, Enterococcus, Morella, Thermoanaerobacterium, or Enterobacter.

In addition, the microorganisms used in the first reaction tank may be used, without limitation, as long as they are capable of producing hydrogen under anaerobic conditions.

In one aspect, a fermentation product in gas phase of the fermentation products produced in the first reaction tank may be introduced into the second reaction tank.

In the second reaction tank, synthesis gas fermentation may be performed, which uses a gas phase, for example, carbon dioxide and hydrogen present externally, to produce organic acids, including acetic acid.

In this case, the carbon dioxide and carbon used in the synthesis gas fermentation may include those present in the atmosphere and may further include carbon dioxide and/or hydrogen included in the gas phase introduced from the first reaction tank.

In addition, in one aspect, the second reaction tank may be fed with hydrogen from a second hydrogen feed tank 70.

Meanwhile, unlike the present invention, when external carbon dioxide is fed during liquid fermentation using organic wastewater without separating liquid fermentation from synthesis gas fermentation, it is difficult to find an appropriate balance between microorganisms involved in liquid fermentation and microorganisms involved in synthesis gas fermentation, resulting in a decrease in acetic acid production efficiency.

In one aspect, the first reaction tank and the second reaction tank may be connected to a microbial production microbial culture device 80. Specifically, a seed microbial cultivator I 81 may be connected to the first reaction tank and a seed microbial cultivator II 82 may be connected to the second reaction tank, and microorganisms stabilized and cultivated in these cultivators may be fed to the first reaction tank and the second reaction tank, so that the fed microorganisms perform fermentation in the first reaction tank and the second reaction tank.

In one aspect, the apparatus for producing biomethane according to the present invention may further include a fourth reaction tank 40 and a hydrogen feed tank 50.

In this case, the gas phase of the fermentation products produced in the third reaction tank may be introduced into the fourth reaction tank.

In one aspect, in the third reaction tank, fermentation may be performed using the fermentation products introduced from the first reaction tank and the second reaction tank.

In one aspect, the fermentation products from the first reaction tank and the second reaction tank may be introduced directly into the third reaction tank, or may be introduced into the third reaction tank via a mixing tank 60. In this case, in the mixing tank, a concentration process of the fermentation product fed to the third reaction tank may be carried out.

The fermentation carried out in the third reaction tank may be anaerobic fermentation. Through the corresponding fermentation, hydrogen, carbon dioxide, and the like are produced, as well as methane.

The carbon dioxide generated in the third reaction tank may be captured by a separate capture device (not illustrated) and used as a substrate for acetic acid fermentation.

In one aspect, the biogas produced in the third reaction tank may be purified and membrane separated to obtain biomethane of high purity, or the gas phase produced in the third reaction tank may be fed to the fourth reaction tank and then subjected to methanation in the fourth reaction tank to obtain biomethane of high purity.

In this case, the microorganisms used in the third reaction tank may be at least one of Methanobacterium, Methanococcus, Methanosarcina, Methanothrix, Methanolobus, Methanococcoides, or Methnoplanus, but are not limited thereto.

In one aspect, the internal temperature of the third reaction tank may be 20 to 60° C., specifically 25 to 60° C., or 25 to 55° C., or 30 to 55° C.

Under the temperature conditions described above, methane production efficiency may be excellent.

In one aspect, the gas phase introduced from the third reaction tank to the fourth reaction tank may include carbon dioxide, methane, and hydrogen.

In addition, the hydrogen feed tank 50 may feed hydrogen to the fourth reaction tank.

In the fourth reaction tank, the gas fed from the third reaction tank and the hydrogen feed tank may be used to produce methane of high purity.

The hydrogen feed tank 50 may feed hydrogen so that the molar fraction (hydrogen:carbon dioxide) of hydrogen and carbon dioxide present in the fourth reaction tank is 3 to 5:1. The hydrogen feed tank may feed hydrogen so that the molar fraction of hydrogen:carbon dioxide in the fourth reaction tank may be, 3.5 to 4.5:1, or 3.8 to 4.5:1, or 3.8 to 4.3:1, or 3.8 to 4.2:1.

The fourth reaction tank may include methane-producing bacteria.

The methane-producing bacteria may include microorganisms capable of producing methane from hydrogen, for example, at least one of Methanobacterials, Methanococcales, Methanomicrobials, or Methanosarcinaceae, but are not limited thereto.

In one aspect, it may be preferred that the methane-producing bacteria be dominant in the fourth reaction tank. For example, it may be preferred to deactivate microorganisms that produce other products that are competitive with the methane-producing bacteria.

The internal temperature of the fourth reaction tank may be 55 to 80° C., specifically, 55 to 75° C., or 60 to 75° C.

In addition, the internal pressure of the fourth reaction tank may be normal pressure to about 10 bar, and a state of being pressurized rather than normal pressure may be preferable with regard to increasing hydrogen solubility.

In one aspect, the fourth reaction tank may be provided with a stirring system (not illustrated) to increase gas solubility. The stirring system may be operated at a speed of about 1000 rpm or less to stir the material in the fourth reaction tank, for example, may stir at 200 rpm or more, 300 rpm or more, 400 rpm or more, 500 rpm or more, 600 rpm or more, 700 rpm or more, 800 rpm or more, or 900 rpm or more, and may stir at 1000 rpm or less, 900 rpm or less, 800 rpm or less, 700 rpm or less, 600 rpm or less, 500 rpm or less, 400 rpm or less, 300 rpm or less, or 200 rpm or less.

In addition, in one aspect, the fourth reaction tank may be provided with a microbubble generator (not illustrated). The microbubble generator may feed gas (e.g., hydrogen) introduced into the fourth reaction tank from the outside of the fourth reaction tank into the fourth reaction tank in the form of microbubbles. The fourth reaction tank is fed with gas in the form of microbubbles through the microbubble generator, which allows improved reactivity between gases (e.g., between hydrogen and carbon dioxide) to be expected.

Under the temperature and pressure conditions described above, methane of high purity may be produced.

In one aspect, a nutrient feed tank may be connected to the fourth reaction tank to provide nutrients to the fourth reaction tank (not illustrated).

In addition, the third reaction tank and the fourth reaction tank may be connected to a sludge storage tank for storing sludge generated in each reaction tank, serving to filter, circulate and feed an unused portion of the sludge to each reaction tank, or feeding organic matter reserves required for methane gas production to the reaction tanks (not illustrated).

In addition, in one aspect, the third reaction tank and the fourth reaction tank may be provided with a discharge pipe for discharging liquid components generated within, or over-introduced into, the respective reaction tanks.

In one aspect, connection pipes may be provided between the respective reaction tanks and feed tanks constituting the apparatus.

In addition, in one aspect, the present invention relates to a system or method for producing biomethane from waste using the apparatus.

As used herein the term “waste” refers to the by-products generated at all stages of the life cycle of life on Earth, including humans and animals, and is used as a broad concept to include materials that are no longer needed for life or activity. For example, the waste includes garbage, combustion residues, excreta, sludge, waste oil, waste acids, waste alkalis, waste materials, animal carcasses, and the like, with no limitation of its form. For example, the waste is a concept that includes all of the liquid phase, such as livestock excreta, the sludge phase, such as food waste, the solid phase, such as waste wood, and the gas phase, such as carbon dioxide emitted by industries.

### DESCRIPTION OF REFERENCE NUMERALS

10: First reaction tank
20: Second reaction tank
30: Third reaction tank
40: Fourth reaction tank
50: Hydrogen feed tank
60: Mixing tank
70: Second hydrogen feed tank
80: Organic acid production microbial culture device
81: Seed microbial cultivator I
82: Seed microbial cultivator II

1. An apparatus for producing biomethane, comprising:
a first reaction tank configured to produce, from a liquid phase including wastewater, a fermentation product including an organic acid;
a second reaction tank configured to produce, from a gas phase, a fermentation product including an organic acid; and
a third reaction tank configured to produce methane from the fermentation product derived from the first reaction tank and the second reaction tank.

2. The apparatus of claim 1, further comprising:
a microbial culture device configured to culture a microorganism and to feed the cultured microorganisms to the first reaction tank.

3. The apparatus of claim 2, wherein the microorganism produces acetic acid or ethanol from carbon dioxide or produces hydrogen under anaerobic conditions.

4. The apparatus of claim 3, wherein the microorganism producing acetic acid or ethanol from carbon dioxide is formed from at least one microorganism selected from the group consisting of Clostridium, Enterobacter, Lactobacillus, Escherichia, Ruminococcus, Propionibacterium, Acetobacterium, Butyribacterium, Streptococcus, and the like.

5. The apparatus of claim 3, wherein the microorganism producing hydrogen under anaerobic conditions is formed from at least one microorganism selected from the group consisting of Clostridium, Sporanaerobacter, Coprococcus, Enterococcus, Morella, Thermoanaerobacterium, and Enterobacter.

6. The apparatus of claim 1, wherein a fermentation product in gas phase of the fermentation products produced in the first reaction tank is introduced into the second reaction tank.

7. The apparatus of claim 6, wherein the gas phase includes at least one of hydrogen or carbon dioxide.

8. The apparatus of claim 1, further comprising:
a mixing tank configured to mix a fermentation product produced in the first reaction tank with a fermentation product produced in the second reaction tank.

9. The apparatus of claim 1, wherein the third reaction tank performs anaerobic fermentation using a microorganism on the fermentation products produced from the first reaction tank and the second reaction tank.

10. The apparatus of claim 9, wherein the microorganism used in the third reaction tank is formed from at least one microorganism selected from the group consisting of Methanobacterium, Methanococcus, Methanosarcina, Methanothhrix, Methanolobus, Methanococcoides, or Methnoplanus.

11. The apparatus of claim 1, wherein an internal temperature of the third reaction tank is 20 to 60° C.

12. The apparatus of claim 1, further comprising:
a fourth reaction tank; and
a hydrogen feed tank, wherein a gas phase of the fermentation products produced in the third reaction tank is introduced into the fourth reaction tank, and wherein the hydrogen feed tank feeds hydrogen to the fourth reaction tank.

13. The apparatus of claim 12, wherein the fourth reaction tank includes methane-producing bacteria.

14. The apparatus of claim 13, wherein the methane-producing bacteria is a microorganism capable of producing methane from hydrogen and is formed from at least one microorganism selected from the group consisting of Methanobacterials, Methanococcales, Methanomicrobials, or Methanosarcinaceae.

15. The apparatus of claim 12, wherein an internal temperature of the fourth reaction tank is 55 to 80° C.

16. The apparatus of claim 12, wherein the hydrogen feed tank feeds hydrogen so that a molar fraction (hydrogen:carbon dioxide) of hydrogen and carbon dioxide present in the fourth reaction tank is 3 to 5:1.

17. The apparatus of claim 12, further comprising:
a microbubble generator coupled to the fourth reaction tank and configured to feed a gas in the form of microbubbles to an interior of the fourth reaction tank.

18. The apparatus of claim 1, wherein the organic acid includes acetic acid.

19. A method of producing biomethane from waste, using the apparatus for producing biomethane according to claim 1.

20. A system for producing biomethane, comprising:
the apparatus for producing biomethane according to claim 1, configured to produce biomethane; and
a sludge storage tank configured to store sludge generated from the apparatus for producing biomethane, filter, circulate and feed an unused portion of the sludge, or feed organic matter reserves required for methane gas production.
